(19) United States
(12) Patent Application Publication
(10) Pub. No.: US 2007/0286834 A1
(43) Pub. Date: Dec. 13, 2007

(54) Water stream dispersible skin care solid composition

(76) Inventors: Alexander G. Popov, St. Petersburg (RU); Vassili G. Popov, Potomac, MD (US)

Correspondence Address: Mr. Vassili G Popov, 7905 Crestdale Drive, Potomac, MD, 20854, US

(21) Appl. No.: 11/450,285

(22) Filed: Jun. 12, 2006

(51) Int. Cl. A61K 8/97; A61K 8/73

(52) U.S. Cl. 424/70.13; 424/74; 424/776

(57) ABSTRACT

PROBLEM TO BE SOLVED: To provide an allergen absorbing solid composition for shower based continuous water streams, that has preventive and alleviative effect by absorbing and removing skin-irritants and allergens.

SOLUTION: This allergen absorbent composition comprises of a powder including one or more component among clay, charcoal, mineral mud. Moreover, the composition includes herbal extracts, essential oils, cellulose ether, binding agent and colorants, when it is necessary.

### CROSS-REFERENCE TO RELATED APPLICATIONS

| Date | Code | Application Number |
|---|---|---|
| n/a | n/a | n/a |

### STATEMENT REGARDING FEDERALLY SPONSORED RESEARCH OR DEVELOPMENT

Current invention not has no relation to any a inventions made under federally sponsored research and development.

### BACKGROUND OF THE INVENTION

Solid shower skin care compositions is the most convenient-in-use and time-saving form of bath-cosmetic and hygienic means. These means allow to consumers to save time for hygienic procedures, but make them more useful.

It is particularly preferred in the today's Ecological situation, when the huge amounts of irritants and allergens everyday attack human skin.

And in this conditions is very significant, what ingredients will be combined into formulation of a shower tablet.

Moreover, many popular and effective skin cleaning materials cause significant drying and wrinkling, allergic reactions and other deleterious effects. There is thus a need for a skin cleaning composition which is effective in cleaning, yet gentle to the skin. There is also a need for a skin cleaning composition which has these properties and, in addition, protects and preserves the skin, supplies antioxidants and minerals to the skin, and requires minimal amounts of preservatives which may cause allergic reactions.

There are descriptions of shower compositions (JP2002241261, JP62283915) having as a base ingredient some soluble inorganic salt, but is not being active and more neutral, than useful, component. And, there are not descriptions of shower cosmetic, applied as base component some anti-allergen and anti-irritants substance.

Thus, it can be seen that a need exists for an improved water dispersible skin care compositions for shower.

Therefore, The objective of this invention is the development of new solid cosmetic preparations for shower, which, in addition to their skin care effects, provide an increased inhibitive effect with regard to inflammation as well as providing a particularly pleasant sensation on the skin.

The invention provides several useful embodiments of shower compositions

### BRIEF SUMMARY OF THE INVENTION

A primary object of the present invention is to provide solid skin care compositions that are being used as dissolvable tablets in the shower and that provides cleaning and anti-irritants effect.

According to the present invention the object of the invention is being achieved by using a high-absorption-activity powdered components such as charcoal, clay or mineral mud, water-soluble polymer is sulfated polyethylene glycol, sulfated carboxy methyl cellulose, herbal extracts, essential oils and, colorants, perfumes and binding agents.

### DETAILED DESCRIPTION OF THE INVENTION

### Field of the Invention

The present invention relates solid dissolvable to cosmetic, aromatic and therapeutic products applied to the skin and used for preparation of cosmetic, aromatic and therapeutic water-based solutions in continuous streams in shower.

### BACKGROUND OF THE INVENTION

Solid shower skin care compositions is the most convenient-in-use and time-saving form of bath-cosmetic and hygienic means. These means allow to consumers to save time for hygienic procedures, but make them more useful. This type of cosmetic composition formed as tablets which are being placed in special containers either in handle of the shower of in wall mounted containers. Shower water stream comes through container and tablet is being dissolved while person is using the shower.

This type of water stream use is extremely effective and is very practical as for for cleaning skin and for treatment of irritants and allergens. It is crucial for this process what kinds of ingredients will be combined into formulation of a shower tablet.

It is important to outline that many popular and effective skin cleaning materials cause significant drying and wrinkling, allergic reactions and other deleterious effects. There is exist a need for a skin cleaning composition which is effective in cleaning, yet gentle to the skin. There is also a need for a skin cleaning composition which has these properties and, in addition, protects and preserves the skin, supplies antioxidants and minerals to the skin, and requires minimal amounts of preservatives which may cause allergic reactions.

There are descriptions of shower compositions (JP2002241261, JP62283915) which are having as a base ingredient some soluble inorganic salt, but is not being active component and is more neutral, than useful component.

And, there are no descriptions of any dissolvable solid shower cosmetic, used as base component of any sort of anti-allergen and anti-irritants substance.

Hence, it can be seen that a need exists for an improved water dispersible skin care compositions for shower.

Therefore, the objective of this invention is the development of new dissolvable solid cosmetic substances for shower, which, in addition to their skin care effects, provide an increased inhibitive effect with regard to inflammation as well as providing a particularly pleasant sensation on the skin.

Current invention provides several useful embodiments of shower compositions.

### SUMMARY AND DETAILED DESCRIPTION OF INVENTION

Among the many substances, which can be used for skin care, the most preferred materials are substances which are having high absorption activity to allergens and skin-irritants, and more effective for improving a physiologic condition of the body. More particularly, which is effective in enhancing the qualities of skin.

Several patents disclose cosmetic compositions, incorporating clays (JP2002167332), charcoal (US2002086066) and powder of mineral mud (U.S. Pat. No. 5,705,172) as a substances, having outstanding absorption activity and high effectiveness as a cleaning and preventive agents. Such materials are well suitable for our purposes as a base ingredients. These patents are may not be considered for use in applications of mentioned ingredients in shower based continuous water streams (SBCWS) because SBCWS have to be created using means of control of the speed of dissolution such as either special form of the tablets or special ingredients for the stabilization of the speed of dissolution.

For the charcoal, for instance, it was discovered that charcoal, when being added to conventional (non-shower based continuous water streams) skin-cleaning compositions, significantly enhances the effectiveness of the skin-cleaning compositions in adsorbing dirt and dead skin cells trapped in the pores of the skin. It is believed that this results from the enhanced ability of the water used along with the cleaning compositions to penetrate the skin. Indeed, it has been found that the addition of charcoal to conventional skin cleaning compositions may also help preserve the skin by promoting moisture retention, promoting blood circulation, and reducing wrinkling.

It is well known, as well, that clay-mask for face provide well cleaning and whitening effect and acts as an anti-allergen and absorbing means.

These base ingredients, may be applied in SBCWS for skin care composition together, as a complex, and separately one from other.

Critically important feature of shower based continuous water streams compositions is availability of additives which are provide means for regulating of solubility speed. In current invention for this purpose is used sulfated polysaccharides, and particularly sulfated carboxy methyl cellulose.

This agent is applied (US2005203055) for treating and preventing various infections, including papilloma virus and fungal and parasitic infections. The carboxy methyl cellulose also relates to use of this substance for the prevention and inhibition of malignant epithelial lesions associated with papilloma virus, such as cervical cancer.

The exact embodiments of the invention are given in claims as formulation

What we claime in our invention is:

1. A water stream dispersible skin care solid composition comprising essentially of 1% to 99% by weight a high-absorption-activity powdered component (HAAPC), from 5% to 40% by weight of water-soluble polymer, 0.5% to 5% by weight of cellulose ether, 0.5% to 7% by weight of herbal extracts, 0.5% to 10% by weight of essential oils. And also suitable colorants, perfumes and binding agents

2. The composition as in claim 1 wherein said HAAPC is clay.

3. The composition as in claim 1 wherein said HAAC is charcoal

4. The composition as claimed in claim 1, wherein said HAAC is mineral mud.

5. The composition as claimed in claim 1, wherein said HAAC is mixture of clay, charcoal and mineral mud.

6. The composition as claimed in claim 1, wherein said water-soluble polymer is polyethylene glycol.

7. The composition as claimed in claim 1, wherein said cellulose ether is carboxy methyl cellulose.

8. The composition as claimed in claim 1, wherein said water-soluble polymer is sulfated polyethylene glycol

9. The composition as claimed in claim 1, wherein said cellulose ether is sulfated carboxy methyl cellulose

10. The composition as in claim 1 comprising essentially of 1% to 90% by weight a high-absorption-activity powdered component (HAAPC), from 90% to 1% by weight of cocoa powder, from 5% to 40% by weight of water-soluble polymer, 0.5% to 5% by weight of cellulose ether, 0.5% to 7% by weight of herbal extracts, 0.5% to 10% by weight of essential oils. And also suitable colorants, perfumes and binding agents
